(19) United States
(12) Patent Application Publication
(10) Pub. No.: US 2025/0198984 A1
(43) Pub. Date: Jun. 19, 2025

(54) ELECTRICAL ASSESSMENT OF DONOR ORGANS

(71) Applicant: Paragonix Technologies, Inc., North Waltham, MA (US)

(72) Inventor: Pratik Patel, North Waltham, MA (US)

(21) Appl. No.: 19/064,519

(22) Filed: Feb. 26, 2025

Related U.S. Application Data
(63) Continuation of application No. PCT/US2023/031193, filed on Aug. 25, 2023.
(60) Provisional application No. 63/401,384, filed on Aug. 26, 2022.
(60) Provisional application No. 63/401,389, filed on Aug. 26, 2022.

(51) Int. Cl. G01N 33/483; A01N 1/126; A01N 1/144; A01N 1/168; G01N 27/02

(52) U.S. Cl. CPC G01N 33/4836; A01N 1/126; A01N 1/144; A01N 1/168; G01N 27/02

(57) ABSTRACT

Systems and methods are provided herein for monitoring electrical activity in ex vivo, resting donor organs. Systems and methods provided herein also include providing electrical stimulation to the donor tissue to maintain or increase viability during storage or transport. Donor organs can include ex vivo, resting hearts. Electrical activity can be used to determine donor organ viability, especially in instances where visual evaluation of a beating heart is not possible. The same electrodes or other sensing devices used to measure electrical activity can be used to apply a voltage/current to the tissue as well in order to maintain viability of the organ. In some embodiments, stimulation or depression may be provided in response to the measured electrical activity.

### INCORPORATION BY REFERENCE TO ANY PRIORITY APPLICATIONS

This application is a continuation of PCT Application US2023/031193, filed Aug. 25, 2023 and titled ELECTRICAL ASSESSMENT OF DONOR ORGANS, which claims the benefit of U.S. Provisional Application No. 63/401,384, filed Aug. 26, 2022, and titled ELECTRICAL ACTIVITY MONITORING IN DONOR ORGANS and U.S. Provisional Application 63/401,389, filed Aug. 26, 2022, and titled ELECTRICAL STIMULATION OF DONOR ORGANS. Each of the aforementioned applications is incorporated by reference herein in its entirety.

### FIELD OF THE INVENTION

The apparatuses, systems, and methods described herein relate to monitoring electrical activity in biological samples, for example organs such as organs intended for donation, and providing electrical stimulation to maintain and improve viability. The recorded electrical activity can be used to determine organ viability and allow for intervention through electrical stimulation in certain embodiments.

### BACKGROUND

There is a critical shortage of donor organs. Hundreds of lives could be saved each day if more organs (heart, kidney, lung, etc.) were available for transplant. While the shortage is partly due to a lack of donors, there is a need for better methods of preserving and transporting donated organs. Within the United States, over 60% of large organs (liver and lung) occur with a donor and recipient who are not local to each other and require that the organ be transported following donation. Current storage and preservation methods allow only a small time window between harvest and transplant, typically on the order of hours. These time windows dictate who is eligible to donate organs and who is eligible to receive the donated organs. These time windows also result in eligible organs going unused because they cannot be transported to a recipient in time. As transport and storage methods have improved, organs can remain viable longer allowing for a wider pool of recipients along with more matches.

One key remaining bottleneck for heart transplantation, however, is the availability of donor hearts for recipients. Approximately 3,800-4,000 successful heart transplants occur every year in the United States. However, the total number of deceased donors in 2021 was ˜13,861 according to UNOS. One of the key reasons for the difference in the number of total deceased donors relative to the actual number of yearly heart transplants is the inability to assess the viability of donor hearts and understandable reluctance to attempt transplantation with a potentially unviable organ. There are a variety of factors that ultimately go into the decision to recover and transplant a donor heart. However, no technology to date has received widespread adoption where the viability of the donor heart is assessed in a clinically meaningful manner during preservation and transport.

There are two general organ donation scenarios. The first is donation after brain death (DBD) and the second is donation after circulatory death (DCD). Most donor hearts in the United States are recovered from DBD cases, with DCD making up a small fraction of the cases. However, the growth of donor hearts for DCD is anticipated to increase given information gained from European experiences and the unmet need for donor hearts for transplantation.

After various clinical assessments are completed to determine the overall match between a donor and recipient, one of the final assessments is gross visualization of the donor heart in sinus rhythm going through the cardiac cycle between diastole and systole. In DBD cases, this gross visualization occurs by the recovery team in-situ in the donor cavity. The DCD cases, this gross visualization occurs after reanimation either 1) in-situ in the case of normothermic regional perfusion (NRP) or 2) on an ex-situ normothermic machine perfusion (NMP) device. In the case of NRP, if the donor heart is deemed acceptable, the organ is cooled down and transported in a non-beating hypothermic state to the recipient. In the case of NMP, the heart is transported while beating in a normothermic manner to the recipient. Lactate levels in NMP have been proposed as a marker for donor heart viability; however, studies have demonstrated that lactate levels may not be a reliable proxy for donor heart viability.

### SUMMARY

Examples/Embodiments described herein relate to monitoring electrical activity in donor tissue including hearts and/or providing electrical stimulation to the donor tissue to maintain or increase viability during storage or transport. Electrical activity can be used to determine donor organ viability, especially in instances where visual evaluation of a beating heart is not possible. The same electrodes or other sensing devices used to measure electrical activity can be used to apply a voltage/current to the tissue as well in order to maintain viability of the organ. Electrical stimulation may be provided at a constant level or may be pulsed similarly to a pacemaker. In some embodiments, stimulation may be provided in response to the measured electrical activity. For example, should the electrical activity drop below a threshold level, a processor in communication with the electrodes can provide a desired current to the preservation fluid or tissue directly to correct the measured drop. In various embodiments, stimulation may also be provided through chemical or mechanical means. For example, should measured electrical activity fall below a threshold, a chemical stimulant such as epinephrine may be delivered to the preservation solution or directly to the donor organ or a compressive force may be applied, optionally in a pulsed manner by, for example, inflating a compressive sleave surrounding the organ.

Accordingly, many organs that might otherwise be deemed unviable for transplant may be rescued and organs that might become unviable during transport may be maintained through applied electrical stimulation, leading to an increased donor pool. Additionally, indications of poor outcomes based on electrical activity monitoring can help avoid those outcomes by preventing the transplant of such organs. Monitoring can be performed in an initial evaluation before transport and/or at transplant and compared to a universal threshold level indicative of a viable organ. In certain embodiments, the threshold may depend on donor or recipient age, sex, fitness, organ size, or other characteristics. In some embodiments viability may be determined by a comparison of electrical activity measured at two or more time points. For example, a measured decrease in electrical activity between recovery and transplant may be used to determine organ viability before transplant. In some embodiments, electrical activity can be measured at fixed time points such as recovery and transplant or may be routinely or constantly measured during transport and recorded for analysis.

Electrical activity can be measured and/or stimulation provided indirectly through sensors in a preservation fluid in which the organ is immersed or may be measured/provided directly via electrodes or sensors placed directly on the organ itself. In some embodiments, recorded electrical activity can be paired with subsequent outcomes for that organ to form a database for subsequent analysis to determine the appropriate thresholds or other patterns in electrical activity indicative of organ viability and positive outcomes. In certain embodiments, that analysis may comprise machine learning analysis with the database of recorded electrical activity and associated outcomes forming a training set for one or more machine learning algorithms (e.g., a neural network).

Monitoring and recording electrical activity can also be used to validate storage and transportation methods by determining which methods best maintain the desired electrical activity indicative of organ viability. Advanced hypothermic preservation of donor hearts that do not subject them to the thermal and physical trauma associated with the decades old standard of ice storage have demonstrated statistically significant benefits in both the immediate postoperative period as well as 1 year survival. Anecdotally, heart transplant surgeons describe the first beat to be more prominent after being preserved using advanced hypothermic preservation compared to donor hearts being preserved with standard ice storage. Clinically, the use of mechanical circulatory support and the overall dose of inotropes during the recovery period has been found to be lower in patients receiving donor hearts using advanced hypothermic storage.

In certain embodiments, intervention in response to measured electrical activity can include not only stimulation to increase activity but, should activity be deemed too high, delivery of cardioplegia to decrease electrical activity. In either instance, interventions can be used to maintain electrical activity within a specified range with the goal of increasing or maintaining organ viability.

The disclosed system and methods can provide a standardized, quantifiable measurement for determining donor organ viability both initially at the donor site as well as during transport and at the transplant location. Furthermore, based on those measurements, embodiments described herein can intervene to stimulate or depress electrical activity. These methods can be used in conjunction with hypothermic transport methods providing real-time monitoring and/or recording of electrical activity for analysis while maintaining a sterile, temperature-stabilized environment for organs during transport. Electrical activity can be added to a suite of monitored parameters including pressure and temperature during transport in order to provide a more complete picture of the organ environment and health before proceeding with transplantation. This differs fundamentally from current state of the art in which DBD and DCD-NRP procured hearts are transported in a static hypothermic state without any insight into the viability of the heart prior to transplant.

Aspects described herein can include a container for transporting a biological sample, the container comprising a sample storage chamber comprising a floor and walls and configured to receive a preservation solution and a biological sample submerged therein, and an electrical system operable to provide electrical stimulation to the biological sample. The electrical system can comprise an electrical sensor operable to measure electrical activity in the biological sample. The electrical system may be submerged in the preservation solution and not in direct contact with the biological sample. The electrical system may be combined with a temperature sensor. The electrical system can be configured to be attached to the biological sample. For example, the electrical system may comprise two or more electrodes that may be attached to the biological sample to provide current therethrough. The electrodes may be configured to attach near one or more of a sinoatrial (SA) node, a atrioventricular (AV) node, a bundle of his, left or right bundle branches, and purkinje fibers of a donor heart.

Containers may include a mesh comprising the two or more electrodes and configured to cover an area of the biological sample. Container may further include a tangible, non-transient memory in electronic communication with the electrical system for recording the electrical activity. The memory may store a threshold level of electrical activity and instructions for the processor to compare the recorded electrical activity to the threshold level. The processor may be operable to provide electrical stimulation to the biological sample through the electrical system when the recorded electrical activity is below the threshold level. The processor may be operable to deliver cardioplegia to the biological sample in the container when the recorded electrical activity is above a threshold level. Container may include a reservoir comprising cardioplegia and operable to deliver the cardioplegia to the biological sample. The reservoir may be in fluidic communication with an adapter to which the biological sample is attached and one or more valves or pumps may be used to control flow of the cardioplegia into the preservation fluid and/or biological sample.

In certain aspects, methods described herein may include maintaining viability of a donor organ for transplantation by delivering electrical stimulation to a donor organ ex vivo with an electrical system. The electrical system can include an electrical sensor, the method further comprising measuring electrical activity in the biological sample. Methods may include comparing the electrical activity in the donor organ to a threshold level and delivering electrical stimulation to the donor organ when the electrical activity is below the threshold level. Method can comprise measuring the electrical activity in the donor organ over time, determining a change in the electrical activity of the donor organ over time, comparing the change in the electrical activity of the donor organ over time to a threshold change, and delivering electrical stimulation to the donor organ when the change in electrical activity is above the threshold level.

In certain embodiments, methods may include submerging the donor organ in a preservation solution and wherein the electrical system is submerged in the preservation solution and not in direct contact with the donor organ. In examples, the method may comprise attaching the electrical system to the donor organ. In some embodiments, methods may include delivering cardioplegia to the donor organ ex vivo. Methods may comprise comparing the electrical activity in the donor organ to a threshold level and delivering cardioplegia to the donor organ when the electrical activity is above the threshold level.

Some embodiments may include a container for transporting a biological sample, the container comprising a sample storage chamber comprising a floor and walls and configured to receive a preservation solution and a biological sample submerged therein; and an electrical sensor operable to record electrical activity in the biological sample. The electrical activity can include voltage. The biological sample may be a heart. The electrical sensor may be submerged in the preservation solution and not in direct contact with the biological sample. In some embodiments, the electrical sensor can be combined with a temperature sensor.

The electrical sensor may be configured to be attached to the biological sample. The electrical sensor can comprise two or more electrodes. The electrodes may be configured to attach near one or more of a sinoatrial (SA) node, a atrioventricular (AV) node, a bundle of his, left or right bundle branches, and purkinje fibers of a donor heart. In some embodiments, the container can comprise a mesh comprising the two or more electrodes and configured to cover an area of the biological sample.

In certain embodiments, containers may further comprise a display on an external surface of the container, in communication with the electrical sensor, and operable to display the electrical activity in the biological sample. Container can include a tangible, non-transient memory in electronic communication with the electronic sensor for recording the electrical activity. The electronic sensor may be in wireless communication with the tangible, non-transient memory. The memory can store a threshold level of electrical activity and instructions for the processor to compare the recorded electrical activity to the threshold level. Container can comprise an alarm in communication with the processor, the processor operable to trigger the alarm when the recorded electrical activity is below the threshold level. Containers can further comprise cooling media which may include a phase change material (PCM) having a phase change formulated between about 0° C. and about 10° C.

Certain aspects may include methods for determining viability of a donor organ for transplantation. Methods may include measuring electrical activity in a donor organ ex vivo with an electrical sensor and determining the donor organ is viable based on the measured electrical activity. In certain embodiments, methods can include comparing the electrical activity in the donor organ to a threshold level wherein the donor organ is viable when the measured electrical activity is above the threshold level. Methods may further comprise measuring the electrical activity in the donor organ over time. A change in the electrical activity of the donor organ over time may be determined and compared to a threshold change wherein the donor organ is viable when the determined change in electrical activity is below the threshold.

Methods can include submerging the donor organ in a preservation solution wherein the electrical sensor is submerged in the preservation solution and not in direct contact with the donor organ. The preservation solution may be between about 0° C. and about 10° C. Methods may further comprise attaching the electrical sensor to the donor organ. The electrical sensor can include two or more electrodes. Methods may include displaying the measured electrical activity on a display in communication with the electrical sensor. The electrical activity can include voltage. The donor organ may be a heart.

### BRIEF DESCRIPTION OF THE DRAWINGS

FIG. 1 is a schematic illustration of a container for storing or transporting a biological sample while monitoring electrical activity and/or delivering electrical stimulation through a preservation solution in which the sample is submerged.

FIG. 2 is an exemplary view of a donor heart with electrodes attached thereto for monitoring of electrical activity and/or providing electrical stimulation.

FIG. 3 is a graph of electrical activity of an ex vivo resting heart showing a potential QRS-like wave after 30 minutes of warm ischemic time.

FIG. 4 is a graph of electrical activity of an ex vivo resting heart showing a potential QRS-like wave after 30 minutes of warm ischemic time and 30 minutes of cold ischemic time.

FIG. 5 is an example flow chart of a method for maintaining electrical activity in an ex-vivo, resting organ.

### DETAILED DESCRIPTION

Embodiments disclosed herein recognize that an acceptable standard for evaluating donor organs, especially hearts, does not currently exist and that, accordingly, potentially viable organs go unused because they cannot be functionally observed. By using electrical sensors, either in a solution in which the organ is submerged (e.g., a preservation solution) or directly attached to the organ, electrical activity can be monitored in the organ at the recovery site, during transportation, and before transplantation. The electrical activity at any given point can be compared to a threshold level indicative of viability. In certain embodiments, electrical activity in the organ can be recorded over time (e.g., during transport) and changes in electrical activity can be analyzed for viability. For example, a significant decrease in electrical activity during transport may indicate that the organ is no longer viable. In some embodiments, the same electrodes or devices used to sense electrical activity (directly or through the preservation solution) may be used to provide electrical stimulation to the organ. In other embodiments, the stimulating electrodes may be separate and/or independent from the electronic sensors.

Past studies have suggested a link poor donor heart storage/transport conditions and conduction abnormalities. In Leonelli et al., 1994, Frequency and significance of conduction defects early after orthotopic heart transplantation, Am J Cardiol., 73(2): 175-9, incorporated herein by reference, a study was conducted in 124 consecutive orthotopic heart transplant patients and found that longer ischemic times were statistically correlated to persistent conduction abnormalities (p=0.04) and that in-hospital mortality was higher in patients with abnormal ECGs versus those with transient or normal ECG (p=0.01). The group concluded that persistent conduction abnormalities more often occurred in donor hearts with ischemic times greater than 160 minutes and that the injury to the conduction system was likely to be a result of ice preservation of the donor heart. Joglar, et al. and Thajudeen, et al. also discussed the relationship between prolonged ischemic time with conduction injury. Thajudeen, et al., 2012, Arrhythmias After Heart Transplantation: Mechanisms and Management, J Am Heart Assoc. 1(2): e001461; Joglar, et al., 2021, Management of arrhythmias after heart transplant: current state and considerations for future research; Circulation: Arrhythmia and Electrophysiology 14.3:e007954, the content of each of which is incorporated herein by reference. Landymore, et al. developed a canine model and concluded through a study with 31 dogs on cardiopulmonary bypass with isoelectric ECG and absence of mechanical activity that small-amplitude electrical activity (10-6V) was present in all dogs at 10° C. Spectral analysis indicated that the frequency of the small-amplitude electrical activity was in the range of 3.25 Hz. In the majority of dogs, delivering cardioplegia did cease small-amplitude electrical activity temporarily, but activity eventually returned. Landymore, et al., 1986, Spectral Analysis of Small-Amplitude Electrical Activity in the Cold Potassium-Arrested Heart, Ann Thorne Surg., 41(4):372-7, incorporated herein by reference.

With an understanding that the organ damage caused by prolonged ischemic times can manifest in negative effects on electrical activity, systems and methods of disclosed herein propose monitoring electrical activity in ex vivo organs such as hearts as a general indicator of organ damage or viability and, in some instances, intervening by providing electrical stimulation to bolster viability. In various embodiments, electrical activity may measured in an initial evaluation before transport. Such applications may be particularly useful in situations where the heart or organ cannot be observed beating in vitro before removal such as in DCD scenarios. An initial evaluation of electrical activity may provide an indicator of whether the heart is viable or not based on comparison of electrical parameters to a threshold or database of parameters from successfully transplanted organs. Accordingly, needed organs that might otherwise go unused can be matched to waiting donors. Electrical parameters can also be measured during transport and/or storage to ensure the organ has remained viable before transplantation and to alert a user to potentially unsuitable conditions what can allow intervention before permanent damage to organ viability occurs.

Monitored electrical activity parameters measured by the electrical sensor can include, for example, voltage and/or frequency. As noted above, such parameters may be compared to a universal threshold level indicative of a viable organ. In some embodiments, the threshold may depend on donor or recipient age, sex, fitness, organ size, or other characteristics. For example, a higher frequency of activity may be expected in a heart from a younger donor.

In some embodiments, the electrical activity of an ex vivo resting organ will be measured. An ex vivo resting organ can be an organ that is not functioning or connected to a living organism. For example, an ex vivo resting organ can be an organ in hypothermic storage. An ex vivo, resting organ can be an arrested heart. For example, an ex vivo resting organ can be an organ in static, hypothermic storage. Methods described herein relate to measuring the electrical activity of an ex vivo resting donor organ to assess viability of the organ. An electrical probe can be used to measure the electrical activity of the ex vivo, resting donor organ. In some embodiments, because the ex vivo resting organ is not functioning, an electrical gain can be used to amplify the electrical signal of the organ. The electrical gain can be 100-10,000 gain. The gain can be approximately 100, 200, 300, 400, 500, 600, 700, 800, 900, 1,000, 1,500, 2,000, 2,500, 3,000, 4,0000, 5,000, 7,000, 7,500, 8,000, 9,000, or 10,000 gain. In a preferred embodiment, the electrical gain can be 1,000 gain. For example, in an ex vivo resting heart, an electrical probe can measure electrical activity that resembles a heart rhythm, including features such as QRS complex. Electrical activity or impedance may be measured to map conduction pathways. Electrical activity or impedance may be measured to sense specialized cells of the heart or other organs. Further, such measures may all for the sensing of newly specialized cells.

The electrical signal of the organ can be measured with a 3-Lead ECG. An electrical probe can be placed near the sinoatrial node. An electrical probe can be placed on the sinoatrial node. An electrical probe can be placed near the apex of the heart. An electrical probe can be placed on the apex of the heart. A reference probe can be placed near the organ. A reference probe can be placed on the organ. The electrical probe can measure electrical activity over time. The electrical probe can measure electrical activity over approximately 30 minutes, 1 hour, 2 hours, 3 hours, 4 hours, 5 hours, 6 hours, 7 hours, 8 hours, or more than 8 hours. Electrical activity over time can be used to determine viability of the organ. Electrical activity over approximately 30 minutes, 1 hour, 2 hours, 3 hours, 4 hours, 5 hours, 6 hours, 7 hours, 8 hours, or more than 8 hours can be used to determine viability of the organ.

The electrical probe can be located near the artery connector of a transport container. The electrical probe can be located near the aortic connector of a heart transport container. The electrical probe can be located near the temperature probe. The electrical probe can measure impedance of the organ. For example, impedance of the organ can be measured after an electrical current is delivered to the organ. The electrical probe can be used to deliver an electrical current to the organ. The electrical probe can measure impedance over approximately 30 minutes, 1 hour, 2 hours, 3 hours, 4 hours, 5 hours, 6 hours, 7 hours, 8 hours, or more than 8 hours. Impedance over time can be used to determine viability of the organ. Impedance over approximately 30 minutes, 1 hour, 2 hours, 3 hours, 4 hours, 5 hours, 6 hours, 7 hours, 8 hours, or more than 8 hours can be used to determine viability of the organ.

In some examples, the electrical probe can be an electrode. The electrical probe can be a micro-electrode array and/or a micro-electrode array on a sleeve. The sleeve can be used to surround the organ at least partially. In certain embodiments, the preservation solution may be sampled to determine electrical activity. The micro-electrode array may be flexible, semi-flexible, or rigid.

In some embodiments viability may be determined by a comparison of electrical activity measured at two or more time points. The electrical sensor may record electrical parameters at regular intervals or when triggered by a user request. In some embodiments, changes in those parameters may be considered and compared to thresholds instead of any one level. For example, a measured decrease in voltage, frequency, or other electrical activity between recovery and transplant may be indicative of organ damage. Depending on the level of the decrease, organ viability may be impacted and the transplant team reviewing the data may choose not to proceed with that organ.

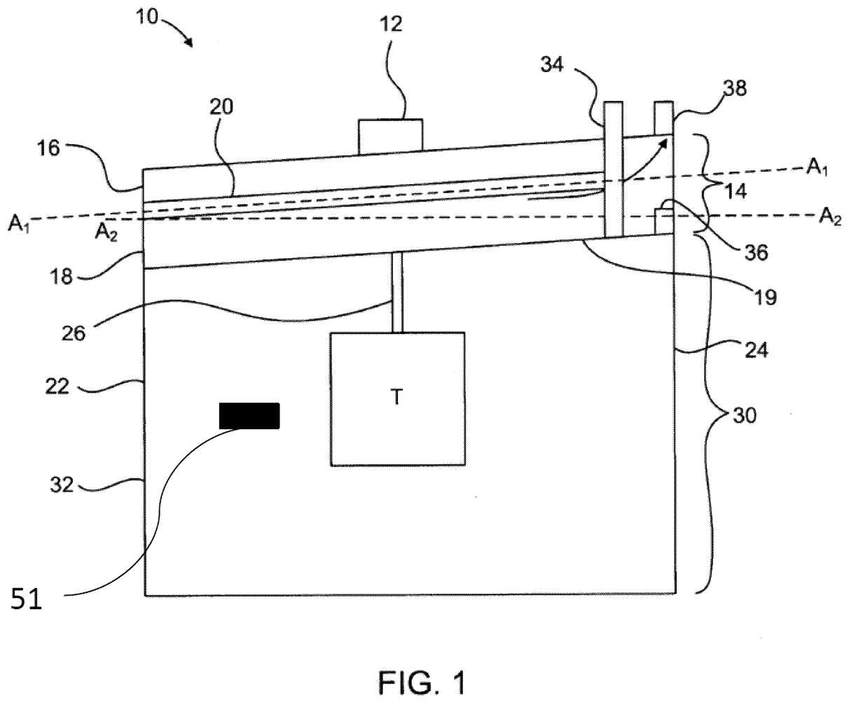

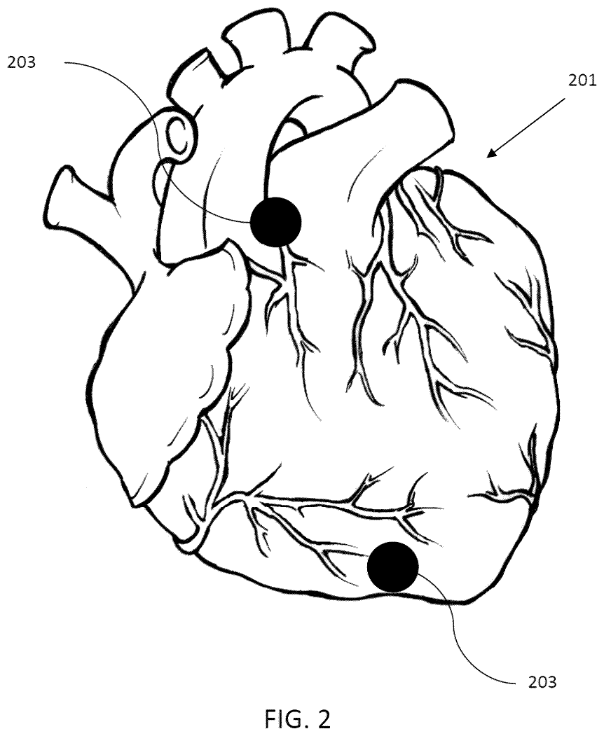

Electrical activity can be measured and/or stimulus provided indirectly through devices in a preservation fluid in which the organ is immersed as shown in FIG. 1 or may be measured directly via electrodes or sensors placed directly on the organ itself as shown in FIG. 2. FIG. 2 illustrates a heart 201 with electrodes 203 attached thereto. The electrical system can comprise two or more electrodes 203 which can be attached near one or more of the sinoatrial (SA) node, the atrioventricular (AV) node, the bundle of his, left or right bundle branches, and the purkinje fibers of a donor heart 201. In some embodiments, an array of electrodes can be positioned on a mesh that can be applied to an area of the organ to provide electrical data and/or stimulation.

Electrical activity can be measured using systems and methods disclosed herein as a research tool to evaluate organs and storage/transport methods. For example, monitoring and recording electrical activity can be used experimentally to validate storage and transportation methods by determining which methods best maintain the desired electrical activity indicative of organ viability. In some embodiments, recorded electrical activity can be paired with subsequent outcomes for that organ to form a database for subsequent analysis to determine the appropriate thresholds or other patterns in electrical activity indicative of organ viability and positive outcomes. In certain embodiments, that analysis may comprise machine learning analysis with the database of recorded electrical activity and associated outcomes forming a training set for one or more machine learning algorithms (e.g., a neural network).

Electrical activity can be added to a suite of monitored parameters including pressure and temperature during transport in order to provide a more complete picture of the organ environment and health before proceeding with transplantation. Electrical sensors disclosed herein may be paired with storage or transport containers designed for hypothermic storage or transport of tissue including donor organs for transplant. As discussed below, a dual temperature/electrical sensor can be used.

In some embodiments, the sensor can be in communication (wirelessly or wired) with a tangible, non-transient memory and a processor. The memory can store a threshold level of electrical activity and instructions for the processor to compare the recorded electrical activity to the threshold level. A change in electrical activity beyond the stored threshold can trigger an alarm or other signal to a user to intervene or may automatically trigger a response in the storage container. Interventions may include confirming an appropriate temperature range, ensuring proper fluid levels, adding chemicals or oxygen to the solution, and, in perfusion apparatuses, checking perfusion status and changing pressures of flow rates of the perfusion fluid. In some embodiments, interventions may include delivering electrical stimulation to the organ to increase electrical activity or providing cardioplegia to decrease or cease electrical activity. To that end, containers may include one or more reservoirs to store cardioplegia. The cardioplegia reservoir can be linked (e.g., via a valved line) to the container interior to provide controlled release of cardioplegia to the preservation solution in which the organ is submerged. In some instances, the cardioplegia can be delivered via a pump and/or to the tissue adapter to that it is delivered in a concentrated form directly to the organ before diluting in the preservation solution generally.

As discussed in more detail below, containers preferably are designed for hypothermic transport and can include cooling media which may comprise a phase change material (PCM) having a phase change formulated between about 0° C. and about 10° C.

Hypothermic transport systems disclosed herein comprise static storage containers and insulated transport containers. Apparatuses disclosed herein can receive tissue for storage or transport, and keep it suspended or otherwise supported temperature controlled, sterile environment and optionally in a surrounding pool of preservation solution. Containers may include self-purging systems where the container is filled with preservation solution and may comprise a number of configurations suitable to transport tissues in a hypothermic manner.

In some embodiments, the storage or transport container will include a pumping mechanism to circulate the preservation solution or perfuse an organ with the preservation solution. A storage or transport container comprising a pumping chamber will be referred to as “pulsatile.” While the pumping is pulsating in preferred embodiments, the pumping is not intended to be limited to pulsating pumping, that is, the pumping may be continuous. In other embodiments, the storage or transport container will not circulate or perfuse the preservation solution. A non-pumping storage or transport container will be referred to as “static.”

In some embodiments, a device is configured to self-purge excess fluid (e.g., liquid and/or gas). For example, in some embodiments, a device includes a lid assembly in which at least a portion of the lid assembly is inclined with respect to a horizontal axis. The inclined portion of the lid assembly is configured to facilitate the flow of fluid towards a purge port disposed at substantially the highest portion of a chamber of the lid assembly. In this manner, excess fluid can escape the device via the purge port. Also in this manner, when excess liquid is expelled from the device via the purge port, an operator of the device can determine that any excess gas has also been purged from the device, or at least from within a tissue chamber of the device, because the gas is lighter than the liquid and will move towards and be expelled via the purge port before excess liquid.

In some embodiments, a device is configured to pump oxygen through a pumping chamber to oxygenate a perfusate and to perfuse a tissue based on a desired control scheme. For example, in some embodiments, the device includes a pneumatic system configured to deliver oxygen to the pumping chamber on a time-based control scheme. The pneumatic system can be configured to deliver oxygen to the pumping chamber for a first period of time. The pneumatic system can be configured to vent oxygen and carbon dioxide from the pumping chamber for a second period of time subsequent to the first period of time. In another example, in some embodiments, the device includes a pneumatic system configured to deliver oxygen to the pumping chamber on a pressure-based control scheme. The pneumatic system can be configured to deliver oxygen to the pumping chamber until a first threshold pressure is reached within the pumping chamber. The pneumatic system can be configured to vent oxygen and carbon dioxide from the pumping chamber until a second threshold pressure is reached within the pumping chamber. In some embodiments, a power source of the device is in use when oxygen is being delivered to the pumping chamber and is not in use when oxygen and carbon dioxide are being vented from the pumping chamber. In this manner, the device is configured to help minimize usage of the power source, and thus the device can prolong the period of time a tissue is extracorporeally preserved within the device before the power source is depleted. Such an improvement increases the time available for transporting the tissue to a hospital for replantation.

As used in this specification, the singular forms “a,” “an” and “the” include plural referents unless the context clearly dictates otherwise. Thus, for example, the term “a fluid” is intended to mean a single fluid or a combination of fluids.

As used herein, “a fluid” refers to a gas, a liquid, or a combination thereof, unless the context clearly dictates otherwise. For example, a fluid can include oxygen, carbon dioxide, or another gas. In another example, a fluid can include a liquid. Specifically, the fluid can be a liquid perfusate. In still another example, the fluid can include a liquid perfusate with a gas, such as oxygen, mixed therein or otherwise diffused therethrough.

As used herein, “tissue” or “biological sample” refers to any tissue of a body of a patient, including tissue that is suitable for being replanted or suspected of being suitable for replantation. Tissue can include, for example, muscle tissue, such as, for example, skeletal muscle, smooth muscle, or cardiac muscle. Specifically, tissue can include a group of tissues forming an organ, such as, for example, the skin, lungs, cochlea, heart, bladder, liver, kidney, or other organ. In another example, tissue can include nervous tissue, such as a nerve, the spinal cord, or another component of the peripheral or central nervous system. In still another example, tissue can include a group of tissues forming a bodily appendage, such as an arm, a leg, a hand, a finger, a thumb, a foot, a toe, an car, genitalia, or another bodily appendage.

Electrical monitoring systems and arrangements described herein are contemplated for use in any variety of static and perfusion devices including self-purging preservations apparatuses as described in U.S. Pat. No. 11,178,866, incorporated herein by reference in its entirety. An exemplary self-purging preservation apparatus 10 according to an embodiment is schematically illustrated in FIG. 1. The self-purging preservation apparatus 10 is configured to oxygenate a perfusate (not shown) received in a pumping chamber 14 of the self-purging preservation apparatus. The self-purging preservation apparatus 10 includes a valve 12 configured to permit a fluid (e.g., oxygen) to be introduced into a first portion 16 of the pumping chamber 14. A membrane 20 is disposed between the first portion 16 of the pumping chamber 14 and a second portion 18 of the pumping chamber. The membrane 20 is configured to permit the flow of a gas between the first portion 16 of the pumping chamber 14 and the second portion 18 of the pumping chamber through the membrane. The membrane 20 is configured to substantially prevent the flow of a liquid between the second portion 18 of the pumping chamber 14 and the first portion 16 of the pumping chamber through the membrane. In this manner, the membrane can be characterized as being semi-permeable.

The membrane 20 is disposed within the pumping chamber 14 along an axis A1 that is transverse to a horizontal axis A2. Said another way, the membrane 20 is inclined, for example, from a first side 22 to a second side 24 of the self-purging preservation apparatus 10. The membrane may be inclined at an angle between 0.5° and 40° relative to horizontal, e.g., between 1° and 30°, e.g., between 5° and 25°, e.g., between 10° and 20°. For example, the membrane may be inclined at an angle between 1° and 10°. As such, as described in more detail below, a rising fluid in the second portion 18 of the pumping chamber 14 will be directed by the inclined membrane 20 towards a port 38 disposed at the highest portion of the pumping chamber 14, thereby allowing the rising fluid to leave the apparatus during filling or during transport. The vent port 38 is configured to permit the fluid to flow from the pumping chamber 14 into the atmosphere external to the self-purging preservation apparatus 10. In some embodiments, the vent port 38 is configured for unidirectional flow, and thus is configured to prevent a fluid from being introduced into the pumping chamber 14 via the port (e.g., from a source external to the self-purging preservation apparatus 10). In some embodiments, the vent port 38 includes a luer lock.

The second portion 18 of the pumping chamber 14 is configured to receive a fluid. In some embodiments, for example, the second portion 18 of the pumping chamber 14 is configured to receive a liquid perfusate. The second portion 18 of the pumping chamber 14 is in fluid communication with an adapter 26. The adapter 26 is configured to permit movement of the fluid from the pumping chamber 14 to a tissue T. For example, in some embodiments, the pumping chamber 14 defines an aperture (not shown) configured to be in fluidic communication with a lumen (not shown) of the adapter 26. The adapter 26 is configured to be coupled to the tissue T. The adapter 26 can be coupled to the tissue Tin any suitable manner. For example, in some embodiments, the adapter 26 is configured to be sutured to the tissue T. In another example, the adapter 26 is coupleable to the tissue T via an intervening structure, such as silastic or other tubing. In some embodiments, at least a portion of the adapter 26, or the intervening structure, is configured to be inserted into the tissue T. For example, in some embodiments, the lumen of the adapter 26 (or a lumen of the intervening structure) is configured to be fluidically coupled to a vessel of the tissue T. In some embodiments, the adapter 26 can comprise an electrical probe. In some embodiments, the adapter 26 can be an artery connector. In some embodiments, the adapter 26 can be an aortic connector.

In some embodiments, the adapter 26 is configured to provide additional support (relieving pressure from the support surface below) to the tissue T when the tissue T is coupled to the adapter. For example, in some embodiments, the adapter 26 includes a retention mechanism (not shown) configured to be disposed about at least a portion of the tissue T and to help retain the tissue T with respect to the adapter and therefore the container. The retention mechanism can be, for example, a net, a cage, a sling, or the like. In some embodiments, the self-purging preservation apparatus 10 includes a support surface configured to support the tissue T when the tissue T is coupled to the adapter 26 or otherwise received in the preservation apparatus 10.

The adapter 26 may be of a variety of structures suitable to suspend the tissue T in the preservation solution while minimizing the potential for mechanical damage, e.g., bruising or abrasion. In some embodiments, the adapter 26 is configured to be sutured to the tissue T. In another example, the adapter 26 is coupleable to the tissue T via an intervening structure, such as silastic or other tubing. In some embodiments, at least a portion of the adapter 26, or the intervening structure, is configured to be inserted into the tissue T. In some embodiments, the adapter 26 is configured to support the tissue T when the tissue Tis coupled to the adapter. For example, in some embodiments, the adapter 26 includes a retention mechanism configured to be disposed about at least a portion of the tissue T and to help retain the tissue T with respect to the adapter. The retention mechanism can be, for example, a net, a cage, a sling, or the like.

In some embodiments, a self-purging preservation apparatus may additionally include a support surface configured to support the tissue T from below wither alone or in conjunction with the adapter 26 when the tissue T is coupled to the adapter 26 or otherwise suspended in the self-purging preservation apparatus. The support mechanism may be part of an insert which fits within the self-purging preservation apparatus. The support surface may include connectors which may be flexible or hinged to allow the support surface to move in response to mechanical shock, thereby reducing the possibility of damage to tissue T. In other embodiments, the support surface may be coupled to the lid assembly so that it is easily immersed in and retracted from the preservation fluid held in the tissue chamber.

A tissue chamber 30 is configured to receive the tissue T and a fluid. In some embodiments, the self-purging preservation apparatus 10 includes a fill port 34 that is extended through the self-purging preservation apparatus 10 (e.g., through the pumping chamber 14) to the tissue chamber 30. The port 34 is configured to permit fluid (e.g., perfusate) to be introduced to the tissue chamber 30. In this manner, fluid can be introduced into the tissue chamber 30 as desired by an operator of the self-purging preservation apparatus. For example, in some embodiments, a desired amount of perfusate is introduced into the tissue chamber 30 via the port 34, such as before disposing the tissue Tin the tissue chamber 30 and/or while the tissue T is received in the tissue chamber. In some embodiments, the fill port 34 is a unidirectional port, and thus is configured to prevent the flow of fluid from the tissue chamber 30 to an area external to the tissue chamber through the port. In some embodiments, the fill port 34 includes a luer lock. The tissue chamber 30 may be of any suitable volume necessary for receiving the tissue T and a requisite amount of fluid for maintaining viability of the tissue T. In one embodiment, for example, the volume of the tissue chamber 30 is approximately 2 liters.

A temperature probe and/or electrical system 51 can be positioned such that the system is submerged in the preservation fluid in the tissue chamber 30 without contacting the tissue T. The electrical system 51 can accordingly be fixed relative to a wall or floor of the tissue chamber 30 at a sufficient distance from the adapter 26 and tissue T. As discussed, the electrical system 51 can comprise an electrical sensor and/or may be coupled to a power source and operable to read electrical activity of and/or deliver electrical current to the tissue T through the fluid. The electrical system 51 can be an electrical probe. The electrical system 51 may be in wireless or wired communication with a display or a processor and memory for displaying and/or recording electrical activity in the tissue T during storage and/or transport. The processor may further control delivery of electrical stimulation to the tissue T. Electrical stimulation may be delivered at a constant rate throughout storage and/or transport in order to increase organ viability. In some instances, electrical stimulation may be variable. The stimulation may be provided and/or its intensity modulated in response to feedback from the electrical or other sensors indicative of naturally occurring electrical activity in the organ or otherwise indicative of organ health. For example, a decrease in measured electrical activity may induce an increased stimulus through the electrical system to maintain organ viability. In various embodiments, stimulation may also be provided through chemical or mechanical means. For example, should measured electrical activity fall below a threshold, a chemical stimulant such as epinephrine may be delivered to the preservation solution or directly to the donor organ or a compressive force may be applied, optionally in a pulsed manner by, for example, inflating a compressive sleave surrounding the organ. Epinephrine or other chemical stimulants may be delivered in a manner similar to that described for cardioplegia herein.

In some embodiments, systems and methods may include at least partial denervation of the organ by, for example, through applied radio frequencies for ablation of target nerves or though chemical means by administering a denervating agent or suppressant into the preservation solution.

The tissue chamber 30 is formed by a canister 32 and a bottom portion 19 of the pumping chamber 14. In a similar manner as described above with respect to the membrane 20, an upper portion of the tissue chamber (defined by the bottom portion 19 of the pumping chamber 14) can be inclined from the first side 22 towards the second side 24 of the self-purging preservation apparatus. In this manner, as described in more detail below, a rising fluid in the tissue chamber 30 will be directed by the inclined upper portion of the tissue chamber towards a valve 36 disposed at a highest portion of the tissue chamber. The valve 36 is configured to permit a fluid to flow from the tissue chamber 30 to the pumping chamber 14. The valve 36 is configured to prevent flow of a fluid from the pumping chamber 14 to the tissue chamber. The valve 36 can be any suitable valve for permitting unidirectional flow of the fluid, including, for example, a ball check valve.

The combination of fill port 34, valve 36, and vent port 38 allow the apparatus to be quickly and reliably filled with preservation fluid during an organ harvest or some other tissue storage procedure. Once the tissue T has been loaded, i.e., with a coupler, sling, or basket as described elsewhere, the pumping chamber 14 can be affixed to the tissue chamber 30, providing an airtight seal. A tube to a reservoir of perfusion fluid can be connected to the fill port 34 allowing the tissue chamber to be filled directly from the outside. Because of the incline of the bottom portion 19 of the pumping chamber 14, any trapped fluids that are less dense than the preservation fluid (e.g., air) will travel along the bottom portion 19 and move to the pumping chamber 14 via valve 36, that can be a one-way check valve. With the addition of more preservation fluid from the fill port 34, the perfusion fluid will also move from the tissue chamber 30 to the pumping chamber 14, driving any less dense fluid to higher points in the pumping chamber 14. When the pumping chamber 14 is finally filled with preservation fluid, all of the rising fluids will be driven out of the apparatus via vent port 38. Thus, a user can simply fill the apparatus via fill port 34 and know that the apparatus is filled with preservation fluid and that all rising fluids (i.e., air) has been driven out of the apparatus when preservation fluid first appears at vent port 38. Additionally, this design conserves preservation fluid ($400/L) when compared to competing designs that immerse an organ in an over-filled preservation fluid, attempting to drive air out of the system as the lid is placed on the device.

The canister 32 can be constructed of any durable materials that are suitable for use with a medical device. For example, it can be constructed of stainless steel. In other embodiments, because it is beneficial to be able to view the contents directly, the lid 6 and storage vessel may be constructed of medical acrylic (e.g., PMMA) or another clear medical polymer. In some embodiments, the canister 32 is constructed of a material that permits an operator of the self-purging preservation apparatus 10 to view at least one of the tissue T or the perfusate received in the tissue chamber 30. For example, in some embodiments, the canister 32 is substantially transparent. In another example, in some embodiments, the canister 32 is substantially translucent. The tissue chamber 30 can be of any suitable shape and/or size. For example, in some embodiments, the tissue chamber 30 can have a perimeter that is substantially oblong, oval, round, square, rectangular, cylindrical, or another suitable shape. Additionally, the self-purging preservation apparatus should be constructed of materials that conduct heat so that the sample within the container is adequately cooled by the cooling media.

It is additionally beneficial for the storage vessel, lid without a pumping chamber, adapter, and any temperature or electrical probes or sensors to be sterilizable, i.e., made of a material that can be sterilized by steam (autoclave) or with UV irradiation, or another form of sterilization. Sterilization will prevent tissues from becoming infected with viruses, bacteria, etc., during transport. In a typical embodiment the self-purging preservation apparatus will be delivered in a sterile condition and sealed in sterile packaging. In some embodiments, the self-purging preservation apparatus will be sterilized after use prior to reuse, for example at a hospital. In other embodiments, the self-purging preservation apparatus will be disposable.

In use, the tissue T is coupled to the adapter 26. The pumping chamber 14 is coupled to the canister 32 such that the tissue T is received in the tissue chamber 30. In some embodiments, the pumping chamber 14 and the canister 32 are coupled such that the tissue chamber 30 is hermetically sealed. A desired amount of perfusate is introduced into the tissue chamber 30 via the port 34. The tissue chamber 30 can be filled with the perfusate such that the perfusate volume rises to the highest portion of the tissue chamber. The tissue chamber 30 can be filled with an additional amount of perfusate such that the perfusate flows from the tissue chamber 30 through the valve 36 into the second portion 18 of the pumping chamber 14. The tissue chamber 30 can continue to be filled with additional perfusate until all atmospheric gas that initially filled the second portion 18 of the pumping chamber 14 rises along the inclined membrane 20 and escapes through the port 38. Because the gas will be expelled from the pumping chamber 14 via the port 38 before any excess perfusate is expelled (due to gas being lighter, and thus more easily expelled, than liquid), an operator of the self-purging preservation apparatus 10 can determine that substantially all excess gas has been expelled from the pumping chamber when excess perfusate is released via the port. As such, the self-purging preservation apparatus 10 can be characterized as self-purging. When perfusate begins to flow out of the port 38, the self-purging preservation apparatus 10 is in a “purged” state (i.e., all atmospheric gas initially within the tissue chamber 30 and the second portion 18 of the pumping chamber 14 has been replaced by perfusate). When the purged state is reached, the operator can close both ports 34 and 38, preparing the self-purging preservation apparatus 10 for operation.

Oxygen (or another suitable fluid, e.g., dry air) is introduced into the first portion 16 of the pumping chamber 14 via the valve 12. A positive pressure generated by the introduction of oxygen into the pumping chamber 14 causes the oxygen to be diffused through the semipermeable membrane 20 into the second portion 18 of the pumping chamber. Because oxygen is a gas, the oxygen expands to substantially fill the first portion 16 of the pumping chamber 14. As such, substantially the entire surface area of the membrane 20 between the first portion 16 and the second portion 18 of the pumping chamber 14 is used to diffuse the oxygen. The oxygen is diffused through the membrane 20 into the perfusate received in the second portion 18 of the pumping chamber 14, thereby oxygenating the perfusate.

In the presence of the positive pressure, the oxygenated perfusate is moved from the second portion 18 of the pumping chamber 14 into the tissue T via the adapter 26. For example, the positive pressure can cause the perfusate to move from the pumping chamber 14 through the lumen of the adapter 26 into the vessel of the tissue T. The positive pressure is also configured to help move the perfusate through the tissue T such that the tissue T is perfused with oxygenated perfusate.

After the perfusate is perfused through the tissue T, the perfusate is received in the tissue chamber 30. In this manner, the perfusate that has been perfused through the tissue T is combined with perfusate previously disposed in the tissue chamber 30. In some embodiments, the volume of perfusate received from the tissue T following perfusion combined with the volume of perfusate previously disposed in the tissue chamber 30 exceeds a volume (e.g., a maximum fluid capacity) of the tissue chamber 30. A portion of the tissue chamber 30 is flexible and expands to accept this excess volume. The valve 12 can then allow oxygen to vent from the first portion 16 of the pumping chamber 14, thus, reducing the pressure in the pumping chamber 14. As the pressure in the pumping chamber 14 drops, the flexible portion of the tissue chamber 30 relaxes, and the excess perfusate is moved through the valve 36 into the pumping chamber 14. The cycle of oxygenating perfusate and perfusing the tissue T with the oxygenated perfusate can be repeated as desired.

A variety of preservation solutions may be used with the embodiments disclosed herein. This includes approved preservation solutions, such as Histidine-Tryptophan-Ketoglutarate (HTK) (e.g., HTK Custodial™) and Celsior™ solutions for the preservation of hearts and cardiac tissues, and University of Wisconsin Solution (Viaspan™) and MPS-1 for the preservation of kidney and kidney tissues. Other preservation solutions, including non-approved solutions, and off-label applications of approved solutions can be used with the devices disclosed herein. Various preservation solutions contemplated for use with embodiments disclosed herein may include Collins, EuroCollins, phosphate buffered sucrose (PBS), University of Wisconsin (UW) (e.g., Belzer Machine Preservation Solution (MPS)), histidine-tryptophan-ketoglutarate (HTK), hypertonic citrate, hydroxyethyl starch, and Celsior™. Additional details of these solutions can be found at t'Hart et al. “New Solutions in Organ Preservation,” Transplantation Reviews 2006, vol. 16, pp. 131-141 (2006), which is incorporated by reference in its entirety. The preservation solution can be a solute, such as a salt solute. An electrical probe can measure electrical activity of an organ through the preservation solution.

Temperature sensors may be any temperature reading device that can be sterilized and maintained in cold environment, i.e., the environment within the container during transport of tissue. The temperature sensor may be a thermocouple, thermistor, infrared thermometer, or liquid crystal thermometer. A temperature display may be coupled to the temperature sensor using any suitable method, for example a wire, cable, connector, or wirelessly using available wireless protocols. As discussed, the temperature sensor and/or housing thereof may be used to house an electrical sensor.

In addition to the temperature sensor and/or electrical sensor, embodiments disclosed herein may include one or more displays. A display can be any display suitable for displaying a temperature measured by the temperature sensor or voltage, frequency, or other information from the electrical sensor, or otherwise provide information about the temperature and/or electrical activity within the self-purging preservation apparatus. For example, the display can be a light emitting diode (LED) display or liquid crystal display (LCD) showing digits corresponding to a measured temperature, recorded voltage, or other electrical parameter. The display may alternatively comprise one or more indicator lights, for example an LED which turns on or off or flashes to indicated whether the temperature measured by the temperature sensor is within an acceptable range, e.g., 2-10° C., e.g., 4-6° C., e.g., about 4° C. and/or whether an electrical parameter is within an acceptable range. Sensors may also be connected to a processor (not shown) which will compare the measured temperature or electrical parameter to a threshold or range and create an alert signal or alarm when the parameter exceeds the threshold or range. The alert may comprise an audible tone, or may signal to a networked device, e.g., a computer, cell phone, or pager that the temperature or electrical parameter within the container exceeds the desired threshold or range.

The container may comprise an insulating material that is effective in maintaining the temperature inside the insulated transport container. A suitable insulating material may be any of a number of rigid polymer foams with high R values, such as polystyrene foams (e.g. STYROFOAM™), polyurethane foams, polyvinyl chloride foams, poly(acrylonitrile)(butadiene)(styrene) foams, or polyisocyanurate foams. Other materials, such as spun fiberglass, cellulose, or vermiculite could also be used. Typically, the insulating vessel will be constructed to provide a close fit for the desired contents (e.g., cooling material/systems, support surface, and organ or other biological sample), thereby affording additional mechanical protection to the tissues contained therein. In some embodiments, the insulated container may be constructed of a closed-cell foam that will prevent absorption of liquids, for example water, body fluids, preservation fluid, saline, etc. In some embodiments, the insulated transport container may include a water-resistant lining (not shown) to facilitate cleaning the insulated transport container after use. In some embodiments, the lining will be removable and disposable. The insulated container may have a hard shell on the exterior to protect the insulating material from damage or puncture. The hard shell may be formed of metal (e.g. aluminum or steel) or of a durable rigid plastic (e.g. PVC or ABS). The hard shell may have antibacterial properties through the use of antibacterial coatings or by incorporation of metal that have innate antibacterial properties (e.g. silver or copper).

The container may have a lid connected thereto with a hinge, hasp, clasp, or other suitable connector. The container lid may also close with a press-fit. The insulated transport container may include an insulating seal to make to make an air-or water-tight coupling between the container and lid. However, the insulated lid need not be sealed to the container for the insulated transport container to maintain a suitable temperature during transport. In some embodiments, the container and lid will be coupled with a combination lock or a tamper-evident device. The container may additionally comprise a handle or a hand-hold or facilitate moving the insulated transport container when loaded. In some embodiments, the container may additionally have external wheels (e.g. castor wheels or in-line skate type wheels). The insulated container may also have a rollaboard-type retractable handle to facilitate moving the system between modes of transport or around a hospital or other medical facility.

The system may use any of a number of active or passive cooling media to maintain the temperature inside the insulated transport container during transport. The cooling media may comprise eutectic cooling blocks, which have been engineered to have a stable temperature between 2-10° C., for example. The cooling media may be arranged in recesses in the interior of the insulated container. The recesses may be a slot or pockets/shelves formed above and under trays or support surfaces as shown in FIG. 2. In certain embodiments, recesses may be a press-fit, or the cooling media may be coupled to the walls, floor, or lid of the container using a snap, screw, hook and loop, or another suitable connecter. Eutectic cooling media suitable for use embodiments disclosed herein is available from TCP Reliable Inc. Edison, NJ 08837, as well as other suppliers. Other media, such as containerized water, containerized water-alcohol mixtures, or containerized water-glycol mixtures may also be used. The container need not be rigid, for example the cooling media may be contained in a bag which is placed in the recess. Using the cooling media, e.g., cutectic cooling blocks, the transport container may be capable of maintaining the temperature of the sample in the range of 2-10° C. for at least 60 minutes, e.g., for greater than 4 hours, for greater than 8 hours, for greater than 12 hours, or for greater than 16 hours.

In various embodiments, cooling blocks may include eutectic cooling media or other phase change material (PCM) such as savENRG packs with PCM-HS01P material commercially available from RGEES, LLC or Akuratemp, LLC (Arden, NC). The PCM may have a phase change formulated between about 0° C. and about 10° C. Exemplary PCM specifications including a freezing temperature of 0° C.+/−0.5° C., a melting temperature of 1° C.+/−0.75° C., latent heat of 310 Jig+/−10 J/g, and density of 0.95 gram/ml +/−0.05 gram/ml. In some embodiments the PCM may have a latent heat of about 200 Jig to about 400 Jig. Pouch dimensions may vary depending on application specifics such as tissue to be transported and the internal dimensions of the transport container and external dimensions of the tissue storage device, chamber, or canister. PCM may be included in pouches approximately 10 inches by 6 inches having approximately 230 g of PCM therein. Pouches may be approximately 8.5 mm thick and weigh about 235 g to 247 g. In some embodiments, pouches may be approximately 6.25 inches by 7.75 inches with a thickness of less than about 8.5 mm and a weight of between about 193 g and about 201 g. Other exemplary dimensions may include about 6.25 inches by about 10 inches. Pouches may be stacked or layered, for example in groups of 3 or 4 to increase the total thickness and amount of PCM. In certain embodiments, PCM containing pouches may be joined side to side to form a band of coupled PCM pouches. Such a band may be readily manipulated to wrap around the circumference of a cylindrical storage container and may have dimensions of about 6 inches by about 26 inches consisting of approximately 8 individual pouches joined together in the band.

The container and portions of the support surface may be constructed from or covered in a sterilizable material, i.e., made of a material that can be sterilized by steam (autoclave) or with UV irradiation, or another form of sterilization. Sterilization will prevent tissues from becoming infected with viruses, bacteria, etc., during transport. In a typical embodiment the sterile canister will be delivered in a sterile condition and sealed in sterile packaging. In some embodiments, the sterile canister apparatus will be re-sterilized prior to reuse, for example at a hospital. In other embodiments, the sterile canister will be disposable.

Thus, using the system for hypothermic transport of tissues, it is possible to transport a biological sample (e.g. tissue, organs, or body fluids) over distances while maintaining a temperature of 2-10° C. Embodiments disclosed herein will enable medical professionals to keep tissues (e.g. organs) in a favorable hypothermic environment for extended periods of time, thereby allowing more time between harvest and transplant. As a result of the embodiments disclosed herein, a greater number of donor organs will be available thereby saving lives.

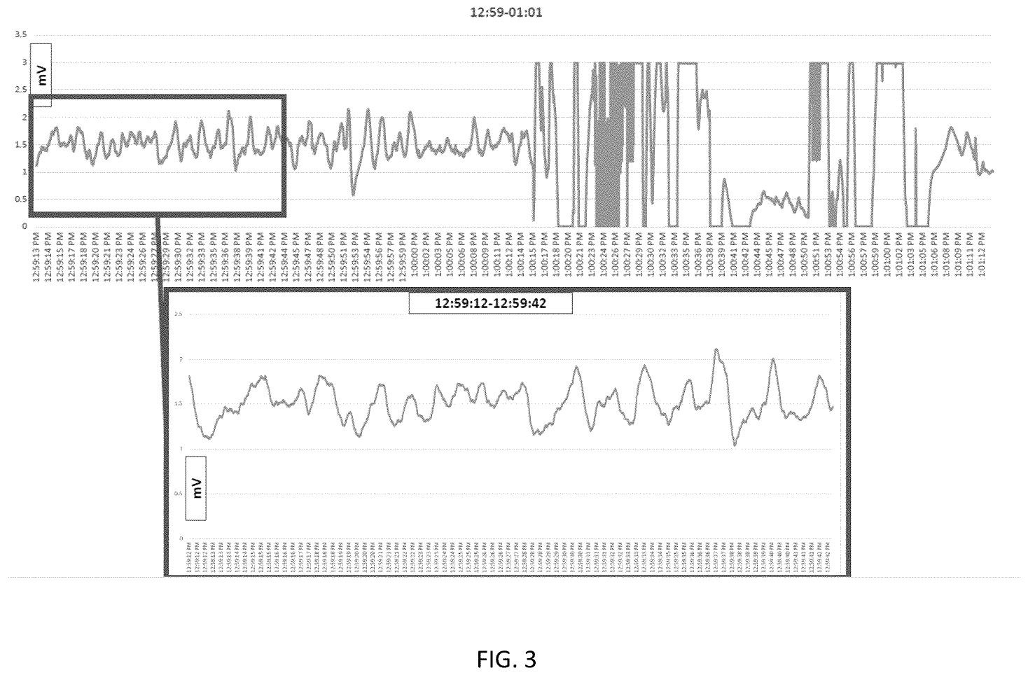

FIG. 3 is a graph of electrical activity in an ex vivo resting heart. The ex vivo resting heart used for the test was a porcine heart excised during necropsy. The porcine heart underwent approximately 25 minutes of total warm ischemic time. A 3-lead ECG with 1,000 Gain was used to measure electrical activity. Electrical probes were placed on the porcine heart near the sinoatrial node and near the apex. A reference probe was also placed near the heart. Electrical signals were measured after another 30 minutes of warm ischemic time. The electrical activity displayed on the graph in mV over time shows a potential QRS-like wave. One of skill in the art will understand that this was a measurement of a single heart and other experiments may result in measurement of additional activity.

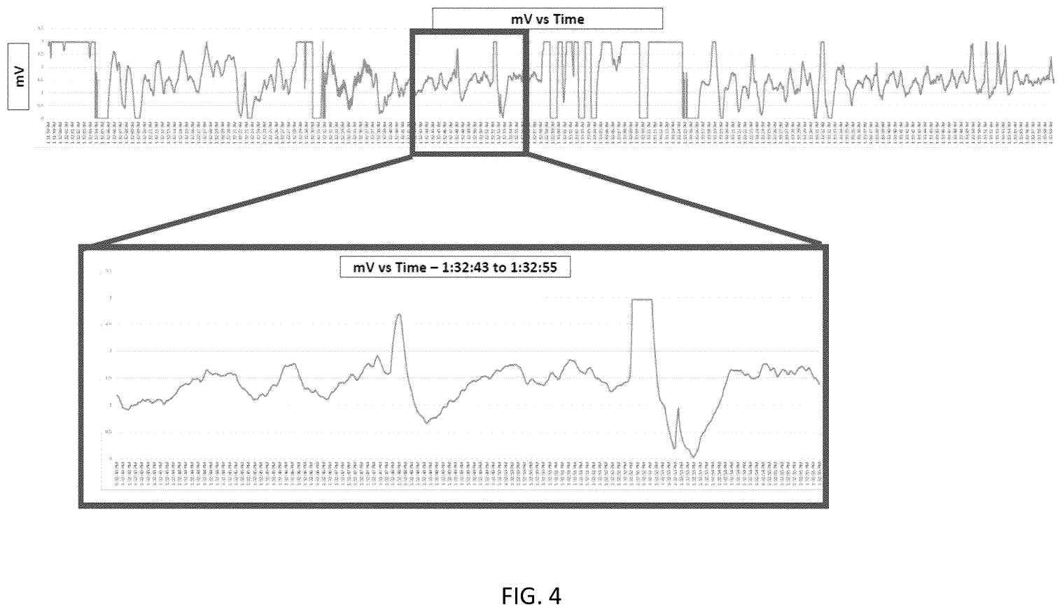

FIG. 4 is a graph of electrical activity in an ex vivo resting heart. The ex vivo, resting heart used for the test was a porcine heart excised during necropsy. The porcine heart underwent approximately 25 minutes of total warm ischemic time. A 3-lead ECG with 1,000 Gain was used to measure electrical activity. Electrical probes were placed on the porcine heart near the sinoatrial node and near the apex. A reference probe was also placed near the heart. Electrical signals were measured after another 30 minutes of warm ischemic time and 30 minutes of cold ischemic time. The porcine heart was placed in 10 mL HTK preservation fluid followed by delivery of 20 V of electricity. The electrical activity displayed on the graph in mV over time shows a potential QRS-like wave. One of skill in the art will understand that this was a measurement of a single heart and other experiments may result in measurement of additional activity.

In some embodiments, the systems and methods herein can include identifying a potential QRS-like wave in an ex vivo resting heart. In some embodiments, the systems and methods herein can include determining viability of an ex vivo resting heart based on a potential QRS-like wave or lack thereof in an ex vivo resting heart.

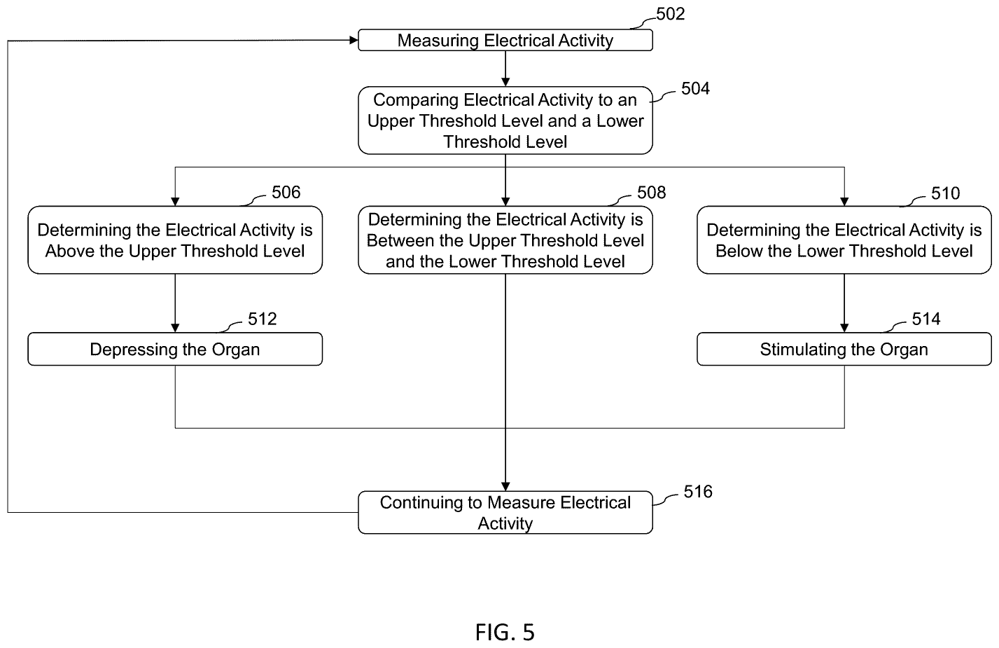

FIG. 5 is an example flow chart of a method for maintaining electrical activity in an ex-vivo, resting organ. The method herein can include measuring electrical activity 502 in a resting, ex-vivo organ with an electrical probe. The method herein can include comparing the electrical activity in the resting, ex-vivo organ to an upper threshold level and a lower threshold level 504. The method herein can include determining that the electrical activity is above the upper threshold level 506. The method herein can include determining that the electrical activity is between the upper threshold level and the lower threshold level 508. The method herein can include determining that the electrical activity is below the lower threshold level 510.

The method herein can include depressing the organ based on the determination that the electrical activity is above the upper threshold level 512. Depressing the organ can include injecting the organ with a chemical to reduce electrical activity. Depressing a heart can include injecting the heart with cardioplegia to reduce electrical activity. The method herein can include stimulating the organ based on the determination that the electrical activity is below the lower threshold level 514. Stimulating the organ can include compressing the organ, injecting the organ with epinephrine, or initiating electrical stimulation. The method herein can include continuing to monitor electrical activity in the organ 516.

### Implementation Consideration

The foregoing description details certain embodiments of the systems, devices, and methods disclosed herein. It will be appreciated, however, that no matter how detailed the foregoing appears in text, the systems, devices, and methods can be practiced in many ways.

As is also stated above, it should be noted that the use of particular terminology when describing certain features or aspects embodiments disclosed herein should not be taken to imply that the terminology is being re-defined herein to be restricted to including any specific characteristics of the features or aspects of the technology with which that terminology is associated.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Headings are included herein for reference and to aid in locating various sections. These headings are not intended to limit the scope of the concepts described with respect thereto. Such concepts may have applicability throughout the entire specification.

Many variations and modifications may be made to the above-described embodiments, the elements of which are to be understood as being among other acceptable examples. All such modifications and variations are intended to be included herein within the scope of this disclosure. The foregoing description details certain embodiments. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

It will also be understood that, when a feature or element (for example, a structural feature or element) is referred to as being “connected”, “attached” or “coupled” to another feature or element, it may be directly connected, attached or coupled to the other feature or element or intervening features or elements may be present. In contrast, when a feature or element is referred to as being “directly connected”, “directly attached” or “directly coupled” to another feature or element, there may be no intervening features or elements present. Although described or shown with respect to one embodiment, the features and elements so described or shown may apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments and implementations only and is not intended to be limiting. For example, as used herein, the singular forms “a”, “an” and “the” may be intended to include the plural forms as well, unless the context clearly indicates otherwise. It will be further understood that the terms “comprises” and/or “comprising,” when used in this specification, specify the presence of stated features, steps, operations, processes, functions, elements, and/or components, but do not preclude the presence or addition of one or more other features, steps, operations, processes, functions, elements, components, and/or groups thereof. As used herein, the term “and/or” includes any and all combinations of one or more of the associated listed items and may be abbreviated as “/”.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features due to the inverted state. Thus, the term “under” may encompass both an orientation of over and under, depending on the point of reference or orientation. The device may be otherwise oriented (rotated 90 degrees or at other orientations) and the spatially relative descriptors used herein interpreted accordingly. Similarly, the terms “upwardly”, “downwardly”, “vertical”, “horizontal” and the like may be used herein for the purpose of explanation only unless specifically indicated otherwise.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, may represent endpoints or starting points, and ranges for any combination of the data points. For example, if a particular data point “10” and a particular data point “15” may be disclosed, it is understood that greater than, greater than or equal to, less than, less than or equal to, and equal to 10 and 15 may be considered disclosed as well as between 10 and 15. It is also understood that each unit between two particular units may be also disclosed. For example, if 10 and 15 may be disclosed, then 11, 12, 13, and 14 may be also disclosed.

Although various illustrative embodiments have been disclosed, any of a number of changes may be made to various embodiments without departing from the teachings herein. For example, the order in which various described method steps are performed may be changed or reconfigured in different or alternative embodiments, and in other embodiments one or more method steps may be skipped altogether. Optional or desirable features of various device and system embodiments may be included in some embodiments and not in others. Therefore, the foregoing description is provided primarily for the purpose of example and should not be interpreted to limit the scope of the claims and specific embodiments or particular details or features disclosed.

The examples and illustrations included herein show, by way of illustration and not of limitation, specific embodiments in which the disclosed subject matter may be practiced. As mentioned, other embodiments may be utilized and derived therefrom, such that structural and logical substitutions and changes may be made without departing from the scope of this disclosure. Such embodiments of the disclosed subject matter may be referred to hercin individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve an intended, practical or disclosed purpose, whether explicitly stated or implied, may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The disclosed subject matter has been provided here with reference to one or more features or embodiments. Those skilled in the art will recognize and appreciate that, despite the detailed nature of the example embodiments provided here, changes and modifications may be applied to said embodiments without limiting or departing from the generally intended scope. These and various other adaptations and combinations of the embodiments provided here are within the scope of the disclosed subject matter as defined by the disclosed elements and features and their full set of equivalents.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The apparatuses, methods, and systems disclosed herein may be embodied in other specific forms without departing from the spirit or essential characteristics thereof. The foregoing embodiments are therefore to be considered in all respects illustrative rather than limiting on the invention described herein. All changes which come within the meaning and range of equivalency of the claims are therefore intended to be embraced therein.

What is claimed is:

1. A method for electrical monitoring of a donor organ, the method comprising:
measuring, using an electrical probe, electrical activity in an ex vivo donor organ during transportation in a transport container while the ex vivo donor organ is in a resting state; and
determining viability of the ex vivo donor organ based at least partially on the electrical activity of the ex vivo donor organ in the resting state during transportation.

2. The method of claim 1, wherein the transport container is a hypothermic static transport container configured to store the ex vivo donor organ within a temperature range of 2-10° C.

3. The method of claim 1, further comprising connecting an artery of the ex vivo donor organ to an artery connector of the transport container, wherein the electrical probe is located near the artery connector.

4. The method of claim 1, wherein the ex vivo donor organ is a heart.

5. The method of claim 4, wherein the heart is placed such that the electrical probe is located near a sinoatrial node of the heart.

6. The method of claim 4, wherein the heart is placed such that the electrical probe is located near an apex of the heart.

7. The method of claim 1, further comprising submerging the ex vivo donor organ in a preservation solution, wherein the electrical probe is submerged in the preservation solution and not in direct contact with the ex vivo donor organ.

8. The method of claim 4, further comprising identifying presence of a potential QRS-like wave in the heart in the resting state.

9. The method of claim 1, wherein viability of the ex vivo donor organ is based at least partially on a change in the electrical activity of the ex vivo donor organ over time in the resting state during transportation.

10. A method for electrical monitoring of a donor organ, the method comprising:
applying a current to an ex vivo donor organ;
measuring, with an electrical probe, impedance of the ex vivo donor organ during transportation in a transport container; and
determining viability of the ex vivo donor organ based on the impedance of the ex vivo donor organ.

11. The method of claim 10, further comprising determining viability of the ex vivo donor organ based on the impedance of the ex vivo donor organ.

12. The method of claim 10, wherein the ex vivo donor organ is placed such that the electrical probe is located near an artery connector of the transport container.

13. The method of claim 10, wherein the ex vivo donor organ is a heart.

14. The method of claim 13, wherein the electrical probe is located near a sinoatrial node of the heart.

15. The method of claim 13, wherein the electrical probe is located near an apex of the heart.

16. The method of claim 10, further comprising submerging the ex vivo donor organ in a preservation solution and wherein the electrical probe is submerged in the preservation solution and not in direct contact with the ex vivo donor organ.

17. The method of claim 10, wherein viability of the ex vivo donor organ is based at least partially on a change in the impedance of the ex vivo donor organ over time during transportation.

18. The method of claim 10, further comprising displaying the impedance of the donor organ on a display of the transport container.

19. A method for electrical monitoring of a donor organ, the method comprising:
providing a transport container containing a donor organ and an electrical sensor;
measuring, using the electrical sensor, electrical activity in the donor organ;
comparing the electrical activity in the donor organ to a plurality of threshold levels;
providing stimulation to the donor organ when the electrical activity is below a threshold level of the plurality of threshold levels; and
providing depression to the donor organ when the electrical activity is above a threshold level of the plurality of threshold levels.

20. The method of claim 19, wherein the donor organ is a heart.

21. The method of claim 19, wherein the stimulation comprises at least one of compression, injection of epinephrine, or electrical stimulation.

22. The method of claim 20, wherein the depression comprises injection of cardioplegia.

23. The method of claim 20, wherein the heart is placed such that the electrical sensor is located near an aortic connector.

24. The method of claim 20, wherein the heart is placed such that the electrical sensor is near at least one of a sinoatrial (SA) node, an atrioventricular (AV) node, a bundle of left or right branches, or purkinje fibers of the donor organ.

25. The method of claim 19, wherein comparing the electrical activity comprises automatically comparing the electrical activity using a processor.

26. The method of claim 19, wherein comparing the electrical activity comprises a user comparing the electrical activity.
